(19) United States
(12) Patent Application Publication
(10) Pub. No.: US 2009/0163902 A1
(43) Pub. Date: Jun. 25, 2009

(54) Cryoprobe With Automatic Purge Bypass Valve

(75) Inventors: Russell L. DeLonzor, Pleasanton, CA (US); Thomas K. Wu, Pleasanton, CA (US)

Correspondence Address: CROCKETT & CROCKETT, P.C., 26020 ACERO, SUITE 200, MISSION VIEJO, CA, 92691, US

(73) Assignee: Sanarus Medical, Inc.

(21) Appl. No.: 11/963,443

(22) Filed: Dec. 21, 2007

(51) Int. Cl. A61B 18/02

(52) U.S. Cl. 606/22

(57) ABSTRACT

A cryosurgical system with a cryoprobe, with a bypass valve communicating between the cryogen supply line and the cryogen exhaust line. The bypass valve is located in the cryoprobe, close to the distal tip of the cryoprobe, and is operable to open upon cryogen flow to purge gasses in the supply pathway and then close automatically when cooled by passing flow of liquid nitrogen.

### FIELD OF THE INVENTIONS

The inventions described below relate the field of cryosurgery.

### BACKGROUND OF THE INVENTIONS

A longstanding problem in the operation of cryoprobes, especially those cooled with liquid nitrogen, is the long delay between the intended initiation of cryogen flow and the actual initiation of flow through the cooling chamber at the tip of the cryoprobe. The delay is due to the long supply hose typically used in liquid cryoprobe systems, and the small diameter of the supply tubing inside the supply/exhaust hose and the inlet tubing inside the cryoprobe itself, and the exhaust tubing in the probe and the supply/exhaust hose, and the propensity of the liquid nitrogen to boil within the supply tubing (before reaching the cryoprobe) and within the inlet and exhaust tubing of the cryoprobe.

The problem of backpressure arising from the boiling of nitrogen within the supply line and cryoprobe tip in various prior art cryosurgical systems is well known. Merry, et al., Apparatus for Cryosurgery, U.S. Pat. No. 4,946,460 (Aug. 7, 1990) proposes to speed cool-down of the liquid nitrogen cryoprobe by diverting liquid nitrogen flow away from the supply hose, several feet upstream from the cryoprobe inlet, and dumping liquid cryogen flow into an evaporator. Goddard, et al., Cryosurgical Instrument, U.S. Pat. No. 5,992,158 (Nov. 30, 1999) provides for venting of gaseous nitrogen from a liquid nitrogen supply line during the first several minutes of nitrogen flow. Gaseous nitrogen is extracted from the flow path in a chamber, and exhaust is regulated by a solenoid-operated vent valve located several feet from the cryoprobe. After several minutes of cryogen flow, the supply hose supply tube and exhaust tube are sufficiently cooled that nitrogen no longer boils with the supply hose, and the vent valve is closed. Both the Merry and the Goddard system appear to result in a significant consumption of liquid nitrogen to overcome boiling-driven backpressure problems. Baust, et al., Cryosurgical Instrument With Vent Means And Method Using Same, U.S. Pat. No. 5,520,682 (May 28, 1996) discloses a cryoprobe with the small vent holes in the probe inlet tube, communicating with the exhaust channel, so that gas within the liquid nitrogen supply line can vent into the exhaust path.

In our co-pending U.S. App. 11/741,524, filed Apr. 27, 2007, entitled Cryosurgical System with Low Pressure Cryogenic Fluid Supply, we disclose a low pressure liquid nitrogen cryoprobe system with numerous modifications designed to minimize cryogen consumption during cryosurgery. The system does not suffer the boiling-driven backpressure problems of the prior art, and even at very low flow rates liquid nitrogen is typically discharged at the exhaust port which is one or two meters proximal to the cryoprobe/supply hose junction. Thus, there is no gaseous cryogen in this supply pathway of this system, and no reason to employ the methods of the prior art to vent such gaseous cryogen in the supply line. A remaining source of delay in the initiation of cooling flow within the cryoprobe cooling chamber is the resistance to flow of the air within the cryoprobe inlet and exhaust tubing, and in the supply and exhaust tubing within the long supply hose. Before cryogen reaches the cooling chamber of the cryoprobe, the cryogen itself must force the air from these significant lengths of tubing.

### BRIEF DESCRIPTION OF THE DRAWINGS

FIG. 1 illustrates a low-pressure liquid nitrogen cryosurgery system.

FIG. 2 is a cross section of the cryoprobe of FIG. 1.

FIG. 3 is a detailed cross section of the cryoprobe of FIG. 1, showing the bypass valve disposed between the cryogen supply/inlet pathway and the cryogen exhaust pathway.

FIGS. 4, 5, 6 and 7 illustrate various constructions of suitable additional bypass valves arrangements in the cryoprobe of FIG. 1.

FIGS. 8 and 9 illustrate a cryoprobe with a bypass valve comprising shape-memory spring, and an opposing biasing spring, disposed between the cryogen supply/inlet pathway and the cryogen exhaust pathway.

FIG. 10 is a cross section of the cryoprobe of FIG. 1, in which the bypass valve is provided in a distinct assembly fitted within the bore of the cross-over fitting.

FIG. 11 is a cross section of the cryoprobe of FIG. 10, in which the bypass valve assembly has been omitted and the feed tube communicates directly with the supply tube through a junction in the crossover fitting.

### DETAILED DESCRIPTION OF THE INVENTIONS

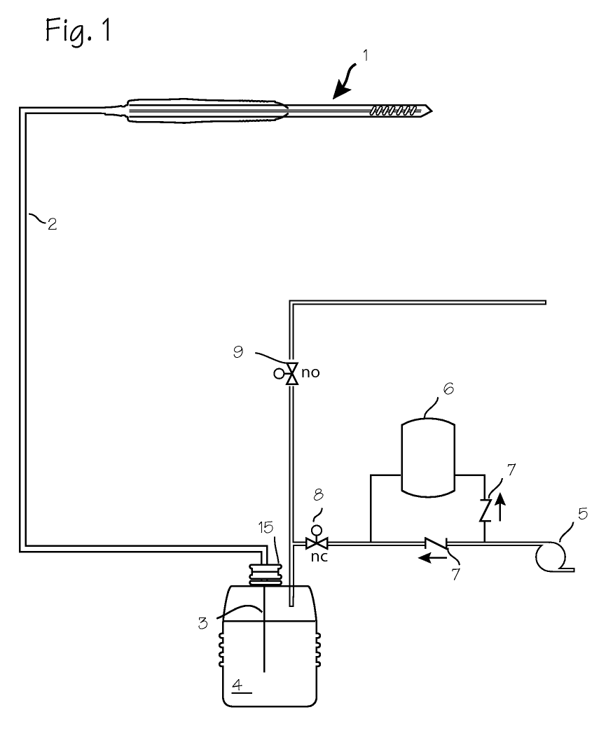

FIG. 1 illustrates the fluid systems of a low-pressure liquid nitrogen cryosurgery system. The cryosurgical system comprises the cryoprobe 1, the supply hose 2 and cryogen supply tube 3 (which runs uninterrupted, within the supply hose, from the Dewar to the probe), a cryogen source Dewar 4, and pressurization pump 5. The desired flow of cryogen from the cryogen source to the cryoprobe is induced by pressurizing the cryogen source with air delivered by the pressurization pump. In this system, the cryogen is supplied in a simple 2 liter (or 2 quart) vacuum-insulated bottle filled with liquid nitrogen (which holds enough liquid nitrogen for numerous procedures), and the pressurization pump is a simple air pump. An accumulator 6 is disposed in parallel between the pressurization pump 5 and the Dewar 4. Check valves 7 prevent backflow from the accumulator to the pump, and pressure supply valve 8 controls flow from either the accumulator or the pump into the Dewar as directed by a control system associated with the fluid system. The pressure supply valve is preferably a solenoid-operated shut-off valve, which is normally closed, but opens when energized to port the output of the pressure pump and accumulator into the Dewar, so that pressurized air is continuously pumped into the Dewar during operation. In normal cryoprobe operation, this valve is maintained open at all times and the pump is operated continuously. (If desired, this valve may be replaced with a throttle valve to be operated to control pressure on the Dewar, but this may increase material requirements of the pressure pump.) To control pressure in the Dewar, the Dewar control valve 9 is operated to bleed pressure off the top of the Dewar. Dewar control valve 9 is a normally open shut-off valve, and is held shut until the Dewar pressure reaches a desired initial pressure and thereafter operated to maintain predetermined steady state operating pressure, by opening and closing the valve at set points just above and below the desired average Dewar pressure to maintain the pressure in a predetermined range, or about a predetermined set point. (The Dewar control valve can also be provided as a throttle valve operated to maintain pressure in the Dewar in the desired range, or pressure relief valve or pressure regulator set to maintain pressure in the Dewar in the desired range.) This arrangement allows the system to be operated with continuous operation of the pump, without the need for a control valve coupled between the pressure source and the liquid Dewar for controlling the pressure supplied to the Dewar.

An associated control system is programmed to operate the fluid system to achieve the cryogen flow desired by the operator, according the method described in our co-pending U.S. App. 11/741,524, filed Apr. 27, 2007 or other treatment regimens. Cryogen flow is initiated when the control system causes the pump and various valves to provide pressure to the Dewar. The various components may operated to pressurize the Dewar to a single set pressure of about 1.5 to 2 bar (about 22 to 30 psi). To provide prompt cooldown of the cryoprobe and speed iceball growth, the fluid system may be operated to provide a slightly higher initial Dewar pressure of about 2.75 to 3.5 bar (about 40 to 50 psi), and thereafter reduce the Dewar pressure to a lower steady state operating pressure of 1.5 to 2 bar (about 22 to 30 psi). For example, the fluid system can be operated to pressurize the Dewar to 40 psi for about 20 seconds, and then slowly reduce the pressure in the Dewar to about 30 psi (over a period of about 40 seconds) by bleeding off pressure from the Dewar through the Dewar control valve and the cryoprobe, and thereafter maintain the pressure in the Dewar at about 30 psi. Steady state pressure may be maintained by opening the Dewar control valve when pressure in the Dewar reaches about 32 psi, and closing the Dewar control valve when the pressure in the Dewar drops to about 28 psi, while operating the pressure pump continuously. This system is our preferred cryogen pressurization system for delivering liquid cryogen from the Dewar and providing pressurized cryogen to the cryoprobe, Other cryogen pressurization means, including cyrogenic pumps, boiling heaters within the cryogen reservoir may be used while still obtaining the benefit of the features described below.

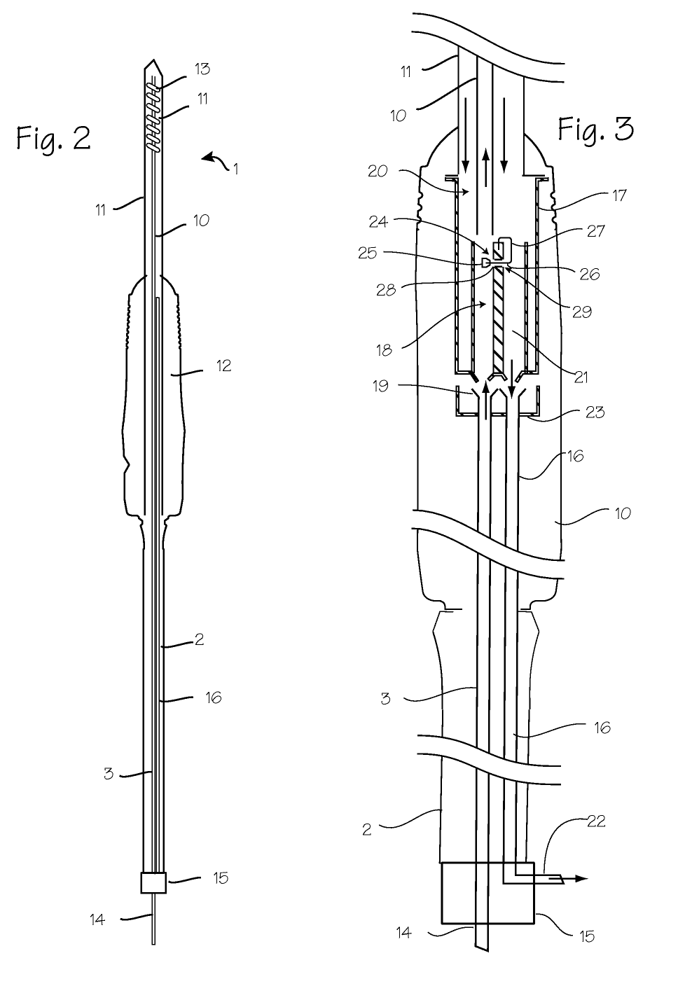

FIG. 2 is a cross section of the cryoprobe of FIG. 1. The cryoprobe 1 comprises an inlet tube 10, a closed-ended outer tube 11, and a handle portion 12. The inlet tube 10 comprises a small diameter tube, and the outer tube comprises a closed end tube, disposed coaxially about the inlet tube. The inlet tube is preferably a rigid tube with low thermal conductivity, such as polyetheretherketone (PEEK, which is well know for its temperature performance), fluorinated ethylene propylene (FEP) or polytetrafluoroethylene. The cryoprobe preferably includes the flow-directing coil 13 or baffle in the cooling segment or boiling chamber at the distal tip of the cryoprobe, disposed coaxially between the inlet tube and the outer tube at the distal end of the cryoprobe. The coil serves to direct flow onto the inner surface of the outer tube, thereby enhancing heat transfer from the outer tube into the cryogen fluid stream. The cryoprobe is described in detail in our co-pending application, DeLonzor, et al., Cryoprobe For Low Pressure Systems, U.S. App, 11/318,142 (filed Dec. 23, 2005), the entirety of which is hereby incorporated by reference. The cryoprobe inlet tube is supplied with cryogen from the cryogen source or Dewar 4 through dip tube 14 and Dewar outlet fitting 15, the supply tube 3 (within the supply hose 2) and the supply hose fitting (see FIG. 1). The dip tube extends from the Dewar outlet fitting 15 fitting into the Dewar, and is preferably a continuous extension of the supply tube 3. The inlet tube 10 may also comprise a continuous distal extension of the supply tube 3, though we currently prefer the construction shown in FIG. 3. The combined length of the dip tube, supply tube, supply side of fitting 17, and the inlet tube constitute a supply pathway for liquid cryogen. An exhaust tube 16, disposed within the supply hose supply hose 2, is connected to the exhaust lumen of the cryoprobe as illustrated in FIG. 3, which together constitute an exhaust pathway.

FIG. 3 is a detailed cross section of the handle of the cryoprobe of FIG. 1, showing the purge bypass valve disposed between the cryogen supply/inlet pathway and the cryogen exhaust pathway. A low-pressure cross-over fitting 17 provides for transition from the coaxial flow of exhaust gas over the inlet tube 10 to the eccentric and side-by-side flow of the supply tube 3 and the exhaust tube 16 in the supply hose, while inducing very little heat introduction into the cryogen flow path. The inlet tube 10, which runs the length of the cryoprobe outer tube, is joined at its proximal end to the supply tube 3 via this low-pressure cross-over fitting. The inlet tube extends proximally through the low pressure fitting and terminates in supply channel 18 which terminates proximally in a frusto-conical nozzle. The swaged (flared) distal end 19 of the supply tube 3 fits over the nozzle to readily form a liquid tight seal. The exhaust flow from the cryoprobe outer tube 11 empties into exhaust plenum 20, and passes through exhaust channel 21 and its frustoconical nozzle into the swaged distal end of the exhaust tube 16, and is eventually exhausted to atmosphere through exhaust vent 22 in or near the Dewar/supply hose fitting. (Though shown separated for clarity, the nozzles and swaged tubes are tightly fitted, and are held in place by the end cap 23 which is tightly secured to the crossover fitting 17.) The preferred material for the inlet tube, supply tube and dip tube is PEEK. The low pressure crossover fitting may comprise PEEK, FEP, nylon or other thermally resistant polymer with very low thermal mass, and may comprise a releasably attachable fitting to join the supply hose to the cryoprobe, or the outer tube to the handle, so that the supply hose can readily be attached and detached from the cryoprobe without use of special tools.

The purge bypass valve 24 provides a flow path for exhausting air from the supply pathway. This valve is held open until liquid cryogen passes the valve, thereby allowing some air to exit the supply pathway through the large valve opening and exhaust tube, and bypass the small inlet tube 10 (some gas in the supply pathway may also be forced into the inlet tube and exhaust through the outer tube 11 and exhaust tube 16). When cold liquid cryogen flow reaches the valve, the valve is closed and the bypass pathway is closed, and the liquid cryogen must flow through the inlet tube. The bypass valves closes automatically when cooled by cryogen flow. The valve plunger 25 is mounted on the valve stem 26, and is driven by a small bi-metallic element 27 (a bi-metallic strip in the illustration) disposed within the exhaust pathway, preferably as near the outlet of the outer tube (or as near the distal end of the supply pathway) as is practical. The bi-metallic strip may also be disposed within the supply pathway, as near the inlet to the inner tube as is practical. At typical ambient temperature, the bi-metallic element holds the stopper off the valve seat established by the aperture 28 and valve bore 29, and when cooled the bi-metallic element acts to pull the stopper into sealing engagement with the valve seat. Thus, as soon as all the air is purged from the supply tube, and liquid cryogen passes through the valve, the valve is shut and cryogen flow is directed to the inlet tube. The valve is thereafter held shut as long as cryogen flow continues, as the exhaust from the exhaust lumen flows over the bi-metallic element. The bypass valve thus operates automatically, without operator or system input, to speed purging upon initial operation and stop purging when purging is substantially complete. When cryogen flow stops and the cryoprobe warms, the bi-metallic strip returns to its warm configuration and opens the valve, so that the cryoprobe will provide bypass flow when cryogen flow is re-initiated.

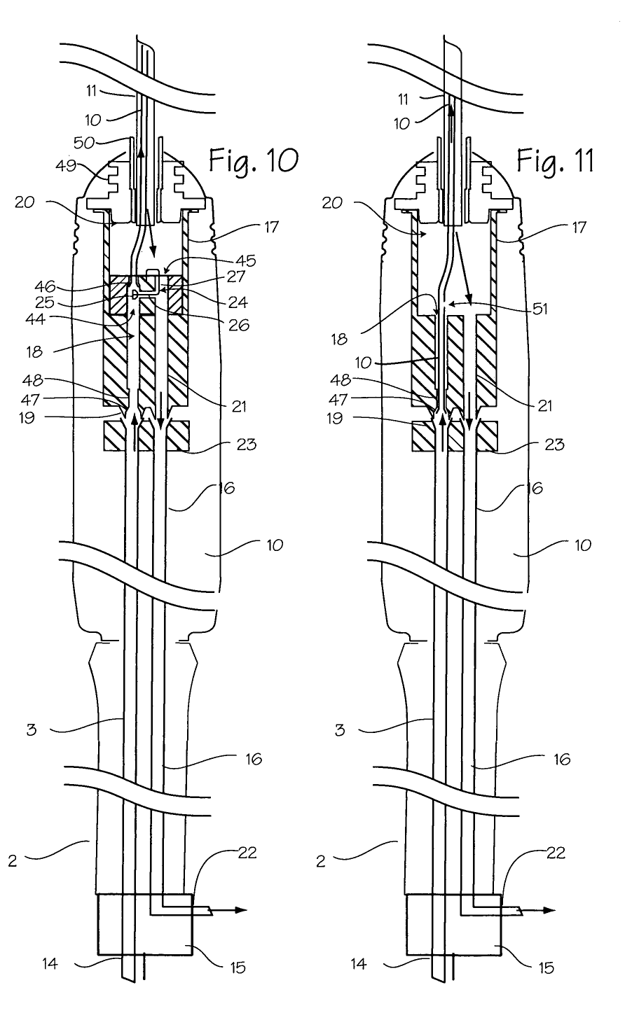

The dimensions of the various components may be varied to achieve desired cryogen flow rates and cooling power. In a system adapted for use treatment of fibroadenomas, with liquid nitrogen supplied at a pressure in the range of 20 to 60 psi (about 1.4 to 4 bar), the dip tube and supply tube 3 may have an internal diameter of 0.075″ (about 1.9 mm) and the inlet tube 10 may have an inner diameter of 0.031″ (about 0.8 mm). The valve bore may have an inner diameter of 0.010″ (about 0.25 mm), and the exhaust channel 21 and exhaust tube 16 may have in internal diameter of 0.111″ (about 2.8 mm). Preferably the exhaust channel 21 and exhaust tube 16 are isodiametric to avoid unnecessary head loss or flow restriction in the exhaust pathway. The supply channel 18 and supply tube 3 may be isodiametric to limit head loss, but the supply pathway may also be varied by introduction of a flow restrictor as discussed below in reference to FIGS. 10 and 11. Operation of the bypass valve in this system results in significant reduction in the time lapse between initiation of cryogen from the Dewar and arrival of cryogen at the probe tip.

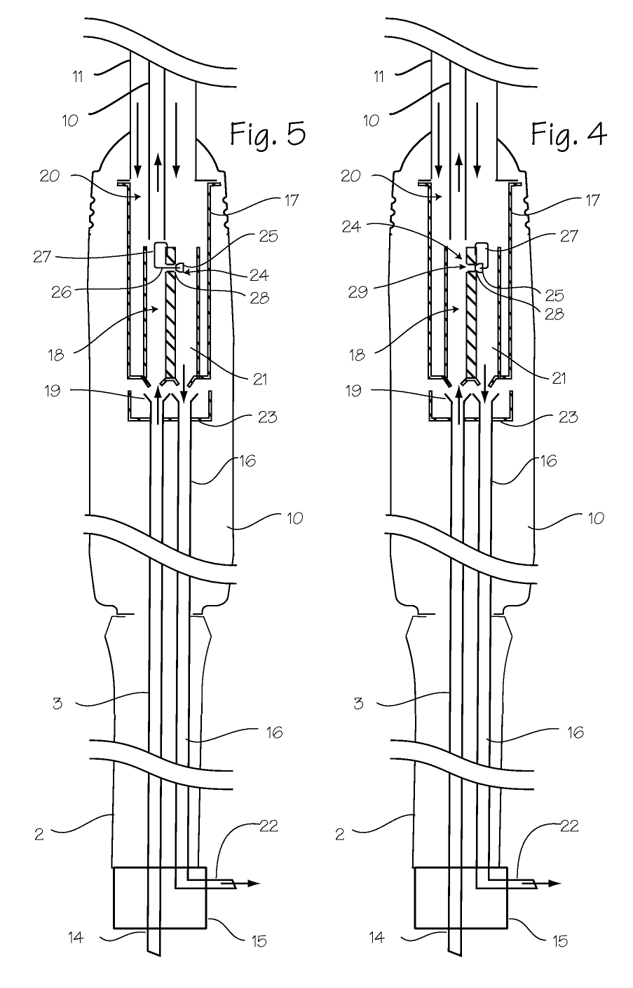

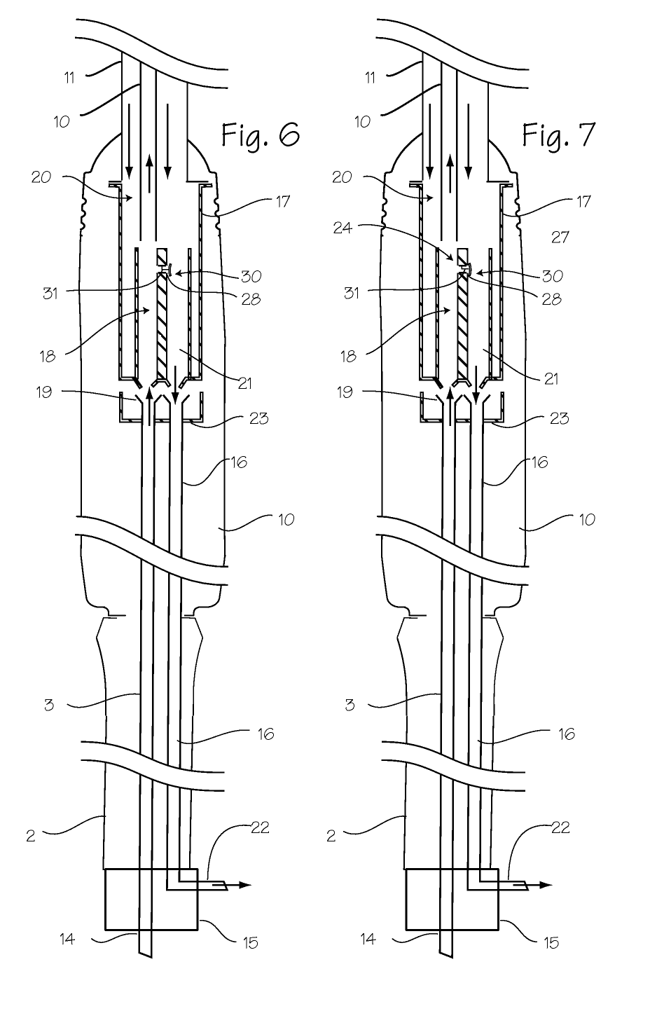

The arrangement of the bi-metallic strip and plunger may be varied. As shown in FIG. 4, the bi-metallic strip 27 may be located in the exhaust pathway, and fixed to the valve plunger 25 located in the exhaust pathway, in which case the bi-metallic strip is arranged to press the valve plunger into the valve seat when cooled to cryogenic temperature. As shown in FIG. 5, bi-metallic strip 27 may be located in the supply pathway (channel 18) and the plunger may be located in the exhaust pathway. A bi-metallic disk may also be used in place of the plunger, stem and strip, as illustrated in FIGS. 6 and 7, where the bi-metallic disk 30 is located over the valve aperture 28, mounted on disk mounting post 31 or otherwise secured proximate the valve seat, such that when cooled it flattens or reverts to a convex shape to occlude the valve aperture and stop bypass flow as shown in FIG. 7.

The bypass valve may comprise other valve structures which operate automatically on changes of temperature, such as thermostatic valve. Other automatic modes of operation may be employed, such as using a solenoid operated valve, or magnetically operated plunger, operated by an associated control system in response to temperature at the inlet tube as sensed by a thermistor or other sensor, or in response to presence of liquid as indicated by a liquid sensor in the flow path near the inlet tube. In each such embodiment, a control system operable to determine the temperature in the exhaust pathway or the presence of liquid in the exhaust pathway (or the distal end of the supply tube, or the inlet tube or the crossover fitting) as indicated by the sensor is operable to shut the valve upon sensing cold fluid.

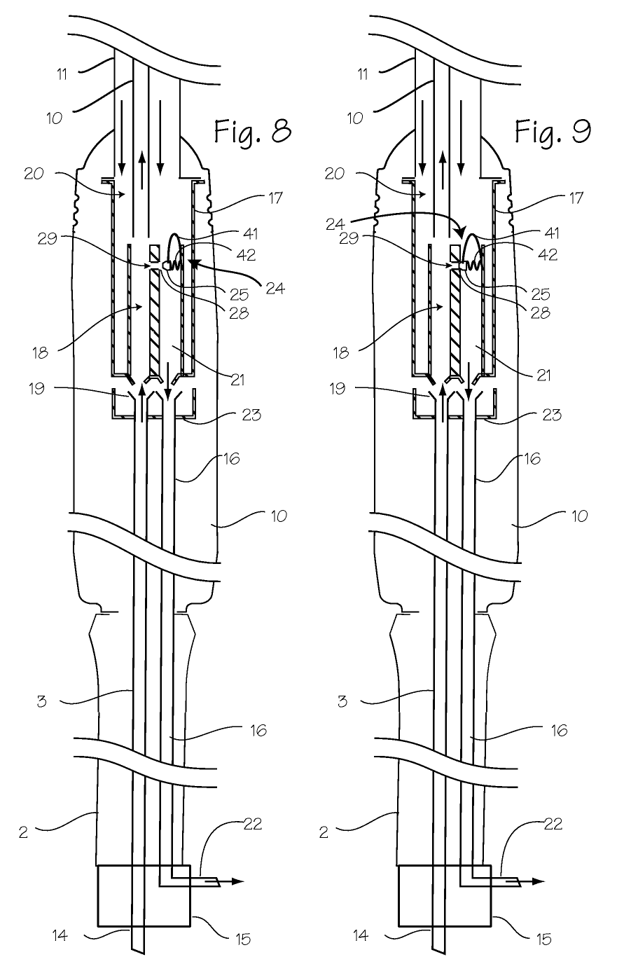

FIGS. 8 and 9 illustrate a cryoprobe with a bypass valve comprising pseudo-elastic or shape memory spring, and an opposing biasing spring, disposed between the cryogen supply/inlet pathway and the cryogen exhaust pathway. The inlet tube 10, the supply tube 3 and exhaust tube 16 and fitting 17 are arranged as described above. The valve actuator in this embodiment comprises a pseudo-elastic actuator 41, in this case a magazine spring, which at high temperature is biased closed (with arms of the magazine spring close to each other) to hold the valve stopper 25 away from the valve bore 29, and a compression spring 42 positioned between the leaves of the magazine spring (or merely impinging or otherwise operable engaged with the stopper) to force the stopper into the valve bore and into sealing position against the valve seat 28. At high temperatures (any temperature above that of the nitrogen exhausting through the exhaust pathways), the magazine spring is austenitic phase, in which the spring is stiff and strong enough to overcome the expansive force of the compression spring and hold the stopper off the valve seat and/or valve bore. This condition is shown in FIG. 8. At low temperature (temperature associated with the passage of nitrogen through the exhaust pathway) the pseudo-elastic magazine spring changes phase to a martensitic phase, in which it is pliable and easily deformed, in which case the compression spring expansive force overcomes the compressive force of the magazine spring to force the stopper into the valve bore to close the valve. The valve actuator remains in the condition, with the valve closed, so long as cryogen is flowing. This condition is shown in FIG. 9.

The actuator of FIGS. 8 and 9 can also comprise opposing leaf springs, one pseudo-elastic leaf spring and one spring steel leaf spring, arranged so that the spring force of the steel leaf spring is directed to force the stopper onto the valve seat, while the spring force of the pseudo-elastic leaf spring in the austenite phase is directed to lift the stopper off the valve seat, and is strong enough to overcome the stainless steel leaf spring. In this embodiment, the pseudo-elastic leaf spring weakens significantly upon cooling, so that the spring steel leaf spring overcomes the pseudo-elastic leaf spring and forces the stopper onto the valve seat. As with the previous embodiments, the valve configuration may be reversed, with the stopper on the inlet side of the valve bore, the pseudo-elastic spring configured to push the stopper and the steel spring configured to pull the stopper, to achieve an equivalent valve actuator. The pseudo-elastic spring is most conveniently made of nitinol or other pseudo-elastic metal, with a martensite transition temperature anywhere between the expected cryogen temperature (−320.44° F. for liquid nitrogen at atmospheric pressure) and just below expected ambient temperatures (to ensure the valve is held open before cryogen flow reached the valve), and an austenite transition temperature anywhere above the steady state temperature of the cryogen, and preferably lower than ambient temperature so that, during the course of a single procedure, the valve may be opened by warming while a surgeon locates additional fibroadenomas for treatment and places the cryoprobe in position. Thus, in successive applications of cryogen using the same probe, the cooling speed is enhanced in each application. For some procedures, only a single placement of the probe is expected, and in probes intended for these procedures, the austenite transition temperature may be immaterial. Other pseudo-elastic materials and shape memory materials, such as shape memory polymers, may be used.

FIG. 10 is a cross section of the cryoprobe of FIG. 1, in which the bypass valve is provided in a distinct assembly fitted within the bore of the cross-over fitting. In this embodiment, the cryogen supply hose, cryogen supply tube, inlet tube 10, cryoprobe outer tube 11, exhaust tube 16, and bypass valve 24 are all as describe above in reference to the earlier figures. The bypass valve components, including the valve stopper 25, valve stem 26, and actuator 27, as illustrated in this figure, are disposed within a bypass valve body 43. The valve body is disposed within the exhaust plenum 20, which is defined by the distal bore of the crossover fitting 17, such that the supply-side bore 44 of the valve body is in fluid communication with the supply channel 18 in the crossover fitting, and the exhaust-side bore 45 of the valve body is in fluid communication with the exhaust channel 21 of the crossover fitting. The crossover fitting outer diameter matches the bore, or inner diameter, of the exhaust plenum. The cross over fitting is glued, tenoned, friction fit, or melted into place within crossover fitting. The inlet tube 10 has a flange, swaged rim, or conical extension on its proximal end. The supply-side bore 44 has a frustoconical portion 46 of the supply-side bore 44 which receives the flange, swaged rim, or conical extension of the inlet tube. The inlet tube curves easily at the bend to enter the center of the outer tube. The proximal portion of the supply channel 18 has a frustoconical portion 47, also sized and dimensioned to receive the flange, swaged rim, or conical extension of the inlet tube, the use of which is described below in reference to FIG. 11. The proximal portion also has a flow restrictor, in the form of a restricted bore, or neck-down portion 48, the dimensions of which may be varied to regulate the delivery conduit, to restrict the flow of cryogen prior to entry into the inlet tube, so as to control the amount of cryogen flowing into the inlet tube from the supply tube. Also shown in FIG. 10 are bushing 49 and 50, for joining the cryoprobe outer tube to the handle 12. The crossover fitting is secured to the bushing with bolts or screws, but may also be glued or melted or snap-fitted to the crossover fitting.

FIG. 11 is a cross section of the cryoprobe of FIG. 10, in which the bypass valve assembly has been omitted and the feed tube communicates directly with the supply tube through a junction in the crossover fitting. The flange, swaged rim, or conical extension of the proximal end of the inlet tube 10 is seated within the frustoconical portion 47. The bypass valve is omitted altogether in this embodiment. Thus, the crossover fitting with frustoconical bore sections at its proximal end may be used either with the valve body as in FIG. 10, or without the valve body, as in FIG. 11, so that a crossover fitting of the same dimensions may be used in various cryoprobe embodiments, and differently sized inlet tubes to be used in conjunction with purge bypass valve bodies can be accommodated by valve bodies provided with appropriately sized conical bores 46. If a degree of bypass is desired, a small aperture 51 may be provided in the side wall of inlet tube, communicating from the interior of the inlet tube to the exterior, preferably the exhaust chamber. This small aperture will allow gaseous air to pass readily, but allow little of the more viscous liquid cryogen to escape, thereby providing some exhaust bypass while resulting in minimal liquid cryogen bypass. This embodiment may be suitable for very small cryoprobes, with outer tubes of about 3 mm or less in outer diameter.

In use, a surgeon inserts the cryoprobe into a patient, with the freezing zone of the cryoprobe outer tube within a lesion. The surgeon confirms the location of the cryoprobe with ultrasound, MRI or other suitable imaging/localization technique. With the cryoprobe properly placed, the surgeon uses the cryogen supply system to initiate cryogen flow from the cryogen source (the Dewar) to the probe. Initially, the inflow of cryogen into the supply pathway displaces air in the pathway. This air is forced through the normally open bypass valve and through the inlet tube of the cryoprobe. When the air is fully purged, the liquid cryogen flows over the valve actuator (or, in boiling systems, cold boiling cryogen flows over the valve actuator), and cools the actuator to the point that the valve closes to cut off bypass flow. In this manner, the bypass flow is cut off when the system is substantially purged without any input or action by the surgeon or control system. IN embodiments using other automatic modes of operation may be employed, such as using a solenoid operated valve, or magnetically operated plunger, operated by an associated control system in response to temperature at the inlet tube as sensed by a thermistor or other sensor, or in response to presence of liquid as indicated by a liquid sensor in the flow path near the inlet tube, the control system operates to shut the valve upon sensing cold fluid near the valve, at any point near the distal end of the supply pathway, without action by the surgeon.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A cryoprobe system comprising:
a cryoprobe comprising an outer tube having proximal end and a closed distal end adapted for insertion into the body of a patient, an inlet tube disposed within the outer tube, said inlet tube having a proximal end and a distal end terminating near the closed distal end of the outer tube, said inlet tube constituting a distal portion of a cryogen supply pathway and said outer tube constituting a distal portion of a cryogen exhaust pathway, and a handle portion secured to the proximal ends of the outer tube and the inner tube;
a reservoir of liquid cryogen and a cryogen pressurization system operable to deliver liquid cryogen from the reservoir;
a cryogen supply tube communicating between the inlet tube and the reservoir of liquid cryogen, said cryogen supply tube constituting a proximal portion of the cryogen supply pathway;
an exhaust tube communicating from the distal end of the outer tube to a point remote from the cryoprobe, said exhaust tube constituting a proximal portion of the cryogen exhaust pathway;
a bypass pathway communicating between the cryogen supply pathway and the exhaust pathway; and
a valve disposed in the bypass pathway, said valve being responsive to temperature such that it closes when cooled by the liquid cryogen, whereby flow of warm fluid residing in the supply tube through the bypass pathway occurs upon initiation of fluid flow into the cryogen supply tube but is stopped when cold cryogen flow reaches the valve.

2. The cryoprobe system of claim 1, wherein the valve further comprises:
a bi-metallic element disposed within the cryogen exhaust pathway or supply pathway, said bi-metallic element fixed to a plunger proximate the bypass pathway, said bi-metallic element having a cooled configuration which forces the plunger into sealing contact with the bypass pathway and a warm configuration which forces into a non-occluding position relative to the bypass pathway.

3. The cryoprobe system of claim 1, wherein the valve further comprises:
a bi-metallic disk disposed within the cryogen exhaust pathway or supply pathway, said bi-metallic disk fixed proximate the bypass pathway, said bi-metallic disk having a cooled configuration which occludes the bypass pathway and a warm configuration which does not occlude the bypass pathway.

4. The cryoprobe system of claim 1, wherein:
a cross-over fitting comprising a supply channel, and exhaust plenum, and an exhaust plenum and an exhaust channel, and said cross-over fitting is interposed between the distal ends of the supply tube and exhaust tube, and the proximal ends of the inlet tube and outer tube, such that the supply channel communicates between the supply tube and the inlet tube, and the outer tube communicates into the exhaust plenum and the exhaust channel communicates between the exhaust plenum and the exhaust tube; and
the bypass pathway communicates between the supply channel and the exhaust channel within the cross over fitting.

5. A method of operating a cryoprobe comprising the steps of:
conducting liquid cryogen through a cryogen supply pathway to a cooling zone of the cryoprobe;
exhausting the liquid cryogen through an exhaust pathway from the cooling zone;
operating a valve disposed between the cryogen supply pathway and the exhaust pathway, maintaining said valve open to allow flow from the cryogen supply pathway to the exhaust pathway while air is purged from the supply line, and closing the valve immediately upon arrival of liquid cryogen at the valve.

6. A cryoprobe comprising:
an outer tube having a closed distal end including a freezing zone for freezing live tissue and a proximal end for receiving liquid cryogenic refrigerant,
a cryogenic refrigerant supply pathway having an inlet for receiving liquid cryogenic refrigerant at the proximal end thereof and a supply pathway outlet for delivering liquid cryogenic refrigerant from the proximal end to the freezing zone at the closed end,
a cryogenic refrigerant exhaust pathway surrounding at least a portion of the supply pathway for transporting the used refrigerant from the closed end towards the proximal end;
vent means in the supply pathway proximal of said freezing zone in fluid communication with the exhaust pathway, thereby enabling during initial operation of the cryoprobe gas present in the supply pathway to be vented through the vent means to the exhaust channel, and disabling during steady state operation of the cryoprobe gas or liquid in the supply pathway to be vented through the vent means to the exhaust channel.
